(19) United States
(12) Patent Application Publication
(10) Pub. No.: US 2001/0014444 A1
(43) Pub. Date: Aug. 16, 2001

(54) Gene regulator fusion proteins and methods of using the same for determining resistance of a protein to a drug targeted thereagainst

(76) Inventors: Laurence M. Melnick, Watertown, MA (US); Donald L. Heefner, Hudson, MA (US)

Correspondence Address: HESLIN & ROTHENBERG, PC, 5 COLUMBIA CIRCLE, ALBANY, NY, 12203

(21) Appl. No.: 09/766,050

(22) Filed: Jan. 19, 2001

Related U.S. Application Data
(63) Continuation of application No. 09/237,821, filed on Jan. 27, 1999, now abandoned.
(60) Provisional application No. 60/093,752, filed on Jul. 22, 1998.
(60) Provisional application No. 60/073,134, filed on Jan. 30, 1998.

(51) Int. Cl. C12Q 1/00; C12Q 1/70; G01N 33/53

(52) U.S. Cl. 435/4; 435/5; 435/7.1

(57) ABSTRACT

Methods and gene regulator fusion proteins are disclosed utilizing a bacterial reporter system to quickly and easily identify mutations of a target protein, such as a protease, that confer resistance to a chemotherapeutic agent directed against that target protein.

### CROSS REFERENCE TO RELATED APPLICATIONS

This application claims the priority of U.S. Provisional Patent Application Ser. Nos. 60/093,752, filed Jul.22, 1998 and 60/073,134, filed Jan. 30, 1998; the entire contents of each is hereby incorporated by reference.

### STATEMENT AS TO RIGHTS UNDER FEDERALLY SPONSORED RESEARCH

This invention was made with support from the National Institutes of Health under NIH Grant No. 1R43 AI38643.01. The United States government may have certain rights in the invention.

### FIELD OF THE INVENTION

The present invention relates to methods for detecting mutations in a protein that confer resistance to a chemotherapeutic agent directed against that protein.

### BACKGROUND OF THE INVENTION

It is well known in the field of drug development that the pathogenicity of various microorganisms, such as viruses, bacteria and the like, may be eliminated, or at least controlled, by inactivating certain proteins essential to the survival and/or proliferation of the microorganisms. One of the more significant scientific and technological advances for the past half-century has been the development of antimicrobial drugs, such as antibiotics and antiviral agents. The widespread availability of these drugs has saved millions of lives and has benefitted mankind in innumerable ways. The only limitation to the usefulness of such drugs has been the evolutionary development of drug resistant microorganisms or pathogens.

Bacterial pathogens may become resistant to antibiotic drugs in a variety of ways, such as by mutating the target of the drug, by limiting uptake of the drug, or by destroying the drug. Often, the drug target is a protein necessary for the survival and/or proliferation of the pathogen, and resistance to the drug is conferred by means of one or more resistance-conferring mutations in the nucleic acid sequence which encodes the drug target. These resistance-conferring mutations result in mutant forms or variants of the drug target protein which retain its functionality but lose its affinity for the drug targeted thereagainst. The problem of widespread and ever-increasing bacterial resistance to antibiotics poses a significant threat to public health, and is the subject of many research efforts throughout the world. See, Harold C. Neu, “The Crisis in Antibiotic Resistance,” Science, 257:1064-1073 (1992).

Bacteria are not the only pathogenic microorganisms that present a problem to the medical community due to their ability to acquire resistance to chemotherapeutic agents or drugs targeted thereagainst. Viruses, most notably the Human Immunodeficiency Virus (“HIV”), present a similar problem with respect to antiviral agents. See, e.g., H. Mohri et al., Proc. Nat'l Acad. Sci., U.S.A., 90:25-29 (1993); M. Tisdale et al., Proc. Nat'l Acad. Sci., U.S.A., 90:5653-5656 (1993); and R. Yarchoan et al., Clinical Perspectives, 14:196-202 (1993).

One of the primary reasons why anti-HIV agents have not been fully effective is the emergence of drug resistance. HIV resistance has been observed for the widely used antiretroviral nucleosides and the HIV protease inhibitors used to treat HIV. With some of these chemotherapeutic agents, resistance has been observed in patients as quickly as six (6) months after treatment has begun. See M. Johnston and D. Hoth, Science, 260:1286-1293 (1993); and M. Waldholz, “Merck faces dismay over test results: HIV resists promising new AIDS drug,” Wall Street Journal (Feb. 25, 1994).

Viral resistant to antiviral agents is typically conferred by one or more resistance-conferring mutations in the viral nucleic acid sequence encoding the targeted viral protein. Particularly in the case of certain retroviruses, such as HIV, as much as twenty percent (20%) of the viruses are found to contain mutations. Wain-Hobson, Current Opinion in Genetics and Development, 3:878-8883 (1993). This high mutational frequency is primarily attributable to the operation of the HIV reverse transcriptase (“RT”) enzyme, which is used to convert single stranded viral RNA into double stranded DNA as part of the viral life cycle but which lacks any editing mechanism. Because of its high mutational frequency, HIV has been characterized as “a perpetual mutation machine”. Id. at 881.

A standard method for attempting to combat drug resistance is the use of HIV whole virus infected cultured cells. For example, serial subculturing in the presence of increasingly higher levels of drugs has led to the in vitro selection of drug resistant HIV variants. Cell culturing is presently being used by a number of groups to detect resistance to candidate HIV protease inhibitory drugs. See, e.g., Jacobsen et al., Meeting abstract “Frontiers in Pathogenesis” Mar. 29 1993, J. Cellular Biochem. Supplement 17E (1993); El-Farrash et al. J. Virol, 68:233-239 (1994); Kaplan et al., Proc. Natl. Acad. Sci., 91:5597-5601 (1994) , Otto et al., Proc. Natl. Acad. Sci., 90: 7543-7547 (1993); and Ho et al., J. Virol, 68:20 6-2020 (1994). Similar cell-culture selection techniques have been used to test the efficacy of antibiotics. See, e.g., Handwerger et al., J. Infectious Dis., 153(1):83 -89 (1986) (wherein clones resistant to benzylpenicillin were selected by serial passage on blood agar plates in two-fold increasing concentrations of benzylpenicillin).

Alternatively, in vitro methods for predicting the identity of all distinct, drug resistant, biologically-active mutants of an original (or “wild-type”) protein that can possible emerge in vivo in response to a chemotherapeutic agent targeted thereagainst has been developed. See PCT International Publication No. WO96/08580, published Mar. 21, 1996, which is incorporated herein by reference in its entirety. These in vitro methods result in extensive variant protein libraries which can then be screened for activity in presence of various chemotherapeutic agents or drugs. These in vitro methods are more rapid, sensitive and free of the bias present in traditional cell culture selection methods. In addition, the resulting library of protein variants can then be screened for susceptibility to various chemotherapeutic agents targeted against that protein.

In one embodiment of the in vitro methods of WO96/08580, an RT-ELISA assay is used for detecting or determining protein, such as HIV protease (“HIV-PR”), drug resistant phenotypes, which assay is described in more detail in WO96/08580. This RT-ELISA assay utilizes E. coli expression of an HIV polyprotein segment including HIV-protease and reverse transcriptase. Activation of RT by the HIV-PR portion of the polyprotein provides the basis for determining HIV-PR drug susceptibility. While this RT-ELISA method for detecting drug resistant protein variants to various chemotherapeutic agents is accurate and useful, it can be somewhat labor intensive and expensive.

### SUMMARY OF THE INVENTION

The present invention relates to gene regulator fusion proteins and methods of using the same for rapidly determining mutations of a protein that confer resistance to a chemotherapeutic agent or drug targeted against that protein.

In one aspect, the present invention relates to a method for detecting mutations in a target protein that confer resistance to a chemotherapeutic agent or drug directed against the target protein, the method comprising the steps of:

(a) preparing random mutations of the gene for the target protein;

(b) subcloning each of the resulting mutant target protein genes into an expression vector or plasmid to form an extended open reading frame encoding a fusion protein including both the target protein and a regulator protein;

(c) preparing a reporter plasmid containing in proper reading sequence a gene for a reporter protein whose activity is regulated by the regulator protein;

(d) introducing the fusion protein expression plasmid from step (b) and the reporter plasmid from step (c) into bacterial cells by electroporation to form a bacterial expression library;

(e) plating the resulting bacterial expression library onto a suitable indicator media containing an amount of a chemotherapeutic agent against the target protein, and incubating the resulting media plates for a period of time; and

(f) identifying from the resulting colonies those colonies which contain drug resistant target protein based on a reporter mechanism of the reporter protein.

In a preferred embodiment, the target protein is HIV-PR, the regulator protein is LacI repressor protein, the reporter protein is β-galactosidase, and the bacterial cells are E. coli.

### BRIEF DESCRIPTION OF THE DRAWINGS

FIG. 1 illustrates the underlying principles of the present invention, in the presence of active target protein. In this embodiment, the fusion protein expression plasmid comprises HIV-PR (target protein) and LacI repressor protein (regulator protein), and the reporter plasmid contains β-galactosidase (reporter protein). The indicator media comprises Xgal substrate (Life Technologies, Inc.).

FIG. 2 illustrates the underlying principles of the present invention, in the absence of active target protein. In this embodiment, the fusion protein expression plasmid comprises HIV-PR (target protein) and LacI repressor protein (regulator protein), and the reporter plasmid contains β-galactosidase (reporter protein). The indicator media comprises XgaI substrate (Life Technologies, Inc.).

In accordance with the principles of the present invention, the presence of a protease inhibitor drug, e.g., indinavir (CRIXIVAN™, Merck & Co.,Inc., Rahway, N.J. USA) thus enables discrimination between drug resistant and drug susceptible HIV-PR variants. In the embodiments illustrated in FIGS. 1 and 2, drug resistant HIV-PR variants will result in white bacterial colonies, while drug susceptible variants will result in blue bacterial colonies.

FIG. 3 is a schematic representation of the random mutagenesis of a target protein gene.

FIG. 4 is a schematic representation of a fusion protein expression plasmid of the present invention. A mutant target protein gene is subcloned into an expression plasmid to form an extended open reading frame encoding a fusion protein including both the mutant target protein gene (e.g. a mutant HIV-PR gene), a regulator protein (e.g. Lacd repressor protein) and an appropriate promoter (e.g. pARABAD, arabinose inducible promoter). On both ends of the mutant target protein gene are the native regions encoding target sites for target protein cleavage. Each expression plasmid of the configuration shown contains a different target protein variant resulting from the mutagenesis depicted in FIG. 3. The fusion protein expression plasmids comprise a library of target protein variants, each attached to a protein which allows reporting of the attached variant.

FIG. 5 is a schematic representation of a reporter plasmid of the present invention. The plasmid contains a reporter protein (e.g. β-galactosidase) and an appropriate promoter (e.g., LacPO, LacI promoter/operator). The expression of the reporter protein is regulated by the regulator protein of the fusion protein expression plasmid.

FIG. 6 illustrates the underlying principles of the fusion protein reporter system of the present invention. A fusion protein expression plasmid of FIG. 4 and a reporter plasmid of FIG. 5 are introduced into bacterial cells (e.g., E. coli) by electroporation to form a bacterial cell expression library which is plated onto a suitable indicator media and incubated. Drug resistant colonies may then be selected based upon the reporter mechanism (e.g., colonies of color A versus colonies of color B) of the reporter protein. DNA may then be isolated from the selected colonies and the DNA sequence of the target protein determined.

### DETAILED DESCRIPTION OF THE INVENTION

An object of the present invention is to proactively determine mutations of a protein target which confer drug resistance to that protein target, thereby enabling the protein target of a chemotherapy to overcome the inhibitory effects of the chemotherapeutic agent being used against the protein target.

The present invention may be used to develop assays for positive selection of drug resistance for a wide range of pathogenic targets of chemotherapy, and to develop chemotherapeutic regimens which are designed to block the evolution by pathogens which lead to drug resistance.

The present invention provides a new method for detecting and identifying mutations in a target protein that confer resistance to chemotherapeutic agents directed against that protein. The basis for the indication of drug susceptibility or resistance is the expression by E. coli cells of a fusion protein consisting of the target protein, a gene regulator protein and a target protein cleavable substrate site located between the target protein and gene regulator protein portions. As a result, activity of the target protein is required to cleave itself from the gene regulator protein, and this cleavage is required in order to activate the regulator protein. In a preferred embodiment, the target protein is HIV-PR.

In one aspect, the method of the present invention involves using a system which includes expression by E. coli of proteins encoded on two distinct plasmids. The first plasmid is induced to express a fusion protein consisting of the target protein, such as HIV-PR, fused to a gene repressor regulator protein, such as LacI. This first plasmid is referred to herein as the “fusion protein expression plasmid”. The second plasmid supplies a reporter protein which provides an indicator of the activity properties of the fusion protein expressed by the first plasmid. This second plasmid is referred to herein as the “reporter plasmid”. For example, the second plasmid may express the E. coli β-galactosidase enzyme configured in the reporter plasmid to be under the regulation of the LacI gene repressor.

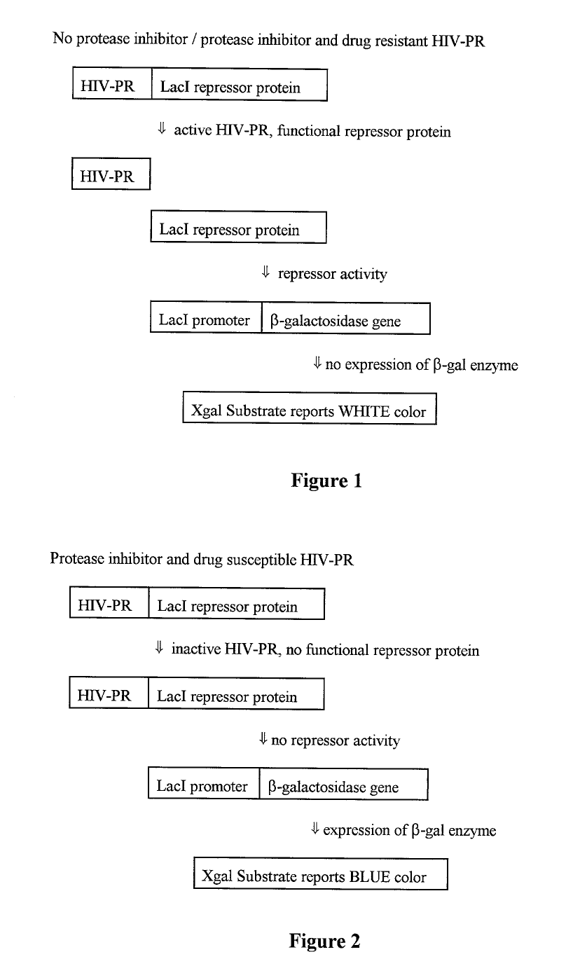

FIGS. 1 and 2 illustrate a method according to the present invention using a two plasmid system that is designed to report on the activity of HIV-PR expressed by E. coli, by using LacI as the regulator protein and β-galactosidase as the reporter protein.

Expression of the E. coli β-galactosidase gene may be readily indicated using the chromogenic substrates Xgal or Bluogal (Life Technologies Inc.), which gives colonies a blue color in the presence of β-galactosidase. The plasmid pUC19 expresses a portion of the β-galactosidase gene required for Bluogal calorimetric report. See, e.g., Davis et al., Basic Methods in Molecular Biology, Elsevier Science Publishing Co., New York, N.Y., 1986, pp 30-31. pUC19 expression of the β-galactosidase segment is “turned off” by LacI repressor protein, thereby giving rise to white colonies on media containing the chromogenic substrate due to the absence of expressed β-galactosidase. In the absence of LacI, β-galactosidase is expressed and the colonies are blue.

The method for determining mutations of a target protein which confer drug resistance to that target protein according to the present invention which uses the two plasmid system is designed to indicate the activity of the target protein (e.g., HIV-PR) expressed by the first plasmid by its effects on the regulation of the expression of β-galactosidase from the second plasmid. As previously stated, the LacI, protein turns off expression of β-galactosidase. Hence, if functional LacI is produced from the first plasmid, then expression of β-galactosidase is turned off and colonies grown on indicator media containing a chromogenic indicator such as Bluogal will not catalyze the formation of a blue product and will appear white. However, if the LacI protein is fused to HIV-PR, its functionality is expected to be compromised and it will not efficiently turn off expression of β-galactosidase from the second plasmid. In this case, E. coli colonies grown on Bluogal indicator media will appear blue, although cleavage of the HIV-PR-LacI fusion protein by the activity of HIV-PR is expected to return function to LacI.

As illustrated in FIGS. 1 and 2, it follows that active fusion proteins containing active HIV-PR give rise to white colonies on indicator media containing Bluogal and fusion proteins containing inactive HIV-PR give rise to blue colonies on such media. Furthermore, inhibitors of HIV-PR should influence the functionality of the LacI in these fusion proteins resulting from the fusion protein expression plasmid by influencing the activity of the HIV-PR component. The influence of protease inhibitors allows discrimination between fusion proteins containing drug susceptible and drug resistant HIV-PR variants.

FIG. 2 illustrates the expected influence of an HIV-PR inhibitor, such as indinavir (CRIXIVAN™, Merck & Co., Inc., Rahway, N.J. USA), on the HIV-PR in E. coli cells containing a fusion protein expression plasmid and a reporter plasmid, wherein a HIV-PR-LacI fusion protein is expressed and β-galactosidase is used as the reporter protein.

According to one embodiment of the present invention, the method for identifying HIV-PR variant genes containing drug resistant mutations comprises the following steps:

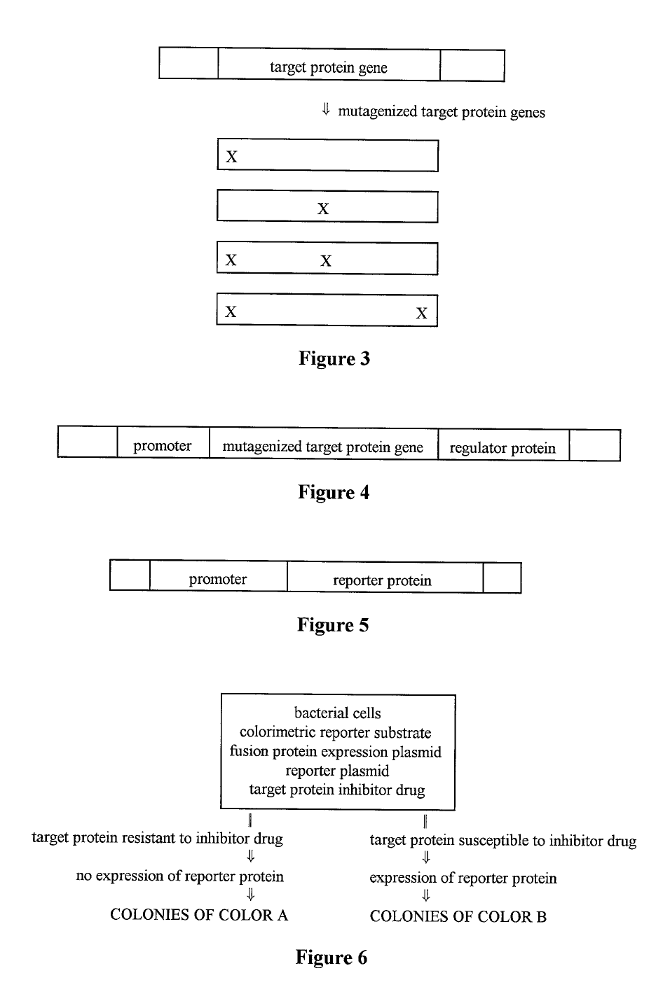

(1) HIV-PR genes containing randomly dispersed mutations are produced using, e.g., error prone PCR (FIG. 3).

(2) The resulting mutant HIV-PR genes are subcloned into an expression vector or plasmid to form an extended open reading frame encoding a fusion protein including both the HIV-PR and the complete LacI where repressor protein, wherein both ends of the HIV-PR gene comprise the native regions encoding target sites for HIV-PR cleavage (FIG. 4). An expression plasmid having this configuration is constructed for each HIV-PR variant resulting from the random mutagenesis of step (1), thereby resulting in a library of fusion protein expression plasmids containing a collection of HIV-PR variants which are each attached to a protein which allows reporting as to the activity of the attached HIV-PR variant.

(3) Each fusion protein expression plasmid, as well as a reporter plasmid containing LacPO and the β-galactosidase genes (FIG. 5), are then introduced into E. coli cells by electroporation to form an E. coli expression library.

(4) The E. coli expression library is then plated onto indicator media comprising antibiotics for maintenance of the plasmids, Bluogal (Life Technologies Inc.) calorimetric reporter substrate for β-galactosidase, arabinose for induction of expression of the HIV-PR containing fusion protein, Isopropyl-β-D-thiogalactopyranoside (“IPTG”) for induction of expression of β-galactosidase, and indinavir (CRIXIVAN™ or MK-639) for inhibition of E. coli expressed drug susceptible HIV-PR.

(5) The E. coli colonies plated onto the indicator media are incubated for approximately sixteen (16) hours, and thereafter white colored colonies, which represent colonies containing drug resistant HIV-PR, are selected and cells from these colonies are grown out in standard media (FIG. 6).

(6) The DNA from the selected E. coli colonies is isolated and the DNA sequence of the drug resistant HIV-PR gene is determined using techniques well-known in the art.

According to one embodiment of the present invention, E. coli cells containing HIV-PR-LacI fusion protein expression plasmids and a reporter plasmid are replica plated onto indicator media containing a protease inhibitor, such as indinavir, saquinavir (INVIRASE™, Roche Laboratories Inc., Nutley, N.J. USA), ritonavir (NORVIR™, Abbott Laboratories, North Chicago, Ill. USA), and nelfinavir (VIRACEPT™, Agouron Pharmaceuticals Inc.).

Although HIV-PR is a preferred target protein for use in methods according to the present invention, this method can be applied to any pathogenic target protein, and in particular pathogenic proteases, for which peptide cleavage sites are defined. The role of maturational protease in vital functions of a wide range of viral pathogens is well known in the art. See, e.g., L. Babe et al., Cell, 91:427-430 (1997). These are excellent alternative chemotherapeutic targets for inclusion in fusion proteins for determination of drug resistant genotypes according to the present invention. In another preferred embodiment, the chemotherapeutic target protein is the hepatitis C virus NS3 serine protease.

A variety of proteins may be used in accordance with the present invention as the regulator protein in the fusion protein in order to activate or repress expression of various bacterial genes or that can function heterologously to express engineered genes in bacteria. For example, the E. coli AraC protein may be used in the present invention.

One skilled in the art would be able to readily determine other chromogenic indicators which may be used in the methods of the present invention. Other indicators of β-galactosidase activity which may be used in accordance with the present invention include, but are not limited to, o-Nitrophenyl-β-D-galactoside (ONPG), methylumbelliferyl-β-D-galactoside (MUG) or Lumi-Gal™ 530 (Lumigen, Inc). See J. Mifiller, A Short Course in Bacterial Genetics, Cold Spring Harbor Press (1992).

The present invention involves methods by which gene regulator fusion proteins can drive positive selections for drug resistant protease variants. In one embodiment, the methods involve regulation by the expressed fusion protein of β-galactosidase expression. In another embodiment, the method can involve the regulation by the expressed fusion protein of the expression of alternative proteins. Gene regulator fusion proteins provide a range of methods for positive selection of drug resistant variants from large libraries of mutants. The term “positive selection” as used herein means a process by which, from among a large library of cells, each expressing a different variant protein(s), only the cells containing the desired, in this case the drug resistant variants, are able to grow. Positive selections eliminate the requirement for plating separated single colonies of bacterial cells for screening and greatly speed up the process of mutation selection.

For example, in the case of positive selection of drug resistant HIV-PR, a growth culture medium may be inoculated with cells such as E. coli, each of which express a different HIV-PR variant. After addition of protease inhibitor to the growth medium and after additional incubation, the culture will only contain cells which express drug resistant HIV-PR variants.

A preferred positive selection method according to the present invention is illustrated by FIGS. 1 and 2 and relates to a method for detecting mutations in HIV-PR that confer resistance to a chemotherapeutic agent directed against that HIV-PR, using a HIV-PR-LacI fusion protein which regulates the expression of the β-galactosidase gene such that, in the presence of a protease inhibitor drug, fusion protein containing drug susceptible HIV-PR fails to produce functional LacI gene repressor. As a result, β-galactosidase is expressed, and on media containing a chromogenic indicator, such as Bluogal, colonies containing the drug susceptible HIV-PR will be easily identified by their blue color (FIG. 2). In contrast, HIV-PR-LacI fusion protein containing drug resistant HIV-PR produces functional LacI, expression of β-galactosidase is repressed, and colonies grown on the indicator media will be easily identified by their white color (FIG. 1).

Another embodiment also involving regulation of β-galactosidase suitable for use in the present invention relates to processing of Phenyl-β-D-galactoside (“Pgal”). In this embodiment, E. coli strains containing GalE mutations are used. When the HIV-PR-LacI fusion protein contains a drug susceptible HIV-PR, β-galactosidase will be expressed, and Pgal is processed by the β-galactosidase to produce a product that is toxic to the E. coli strains containing GalE mutations. Thus, only colonies containing drug resistant HIV-PR-LacI fusion proteins will remain.

A similar result can be achieved by modifying the regulator plasmid to contain a strong promoter which results in β-galactosidase overexpression in drug susceptible HIV-PR containing cells which is toxic to E. coli cells. Moreover, overexpression of a wide range of proteins in E. coli in addition to β-galactosidase, including many viral and mammalian proteins, is toxic to the E. coli cells. Hence, regulation of the expression of such proteins by gene regulator fusion protein in accordance with the present invention can drive positive selections of drug resistant protease variants.

According to the present invention, the reporter plasmid can also be modified to replace the β-galactosidase gene with a gene for a toxic protein the expression of which is engineered to be regulated by the LacI repressor protein from the fusion protein. The toxic proteins for use in the present invention include, but are not limited to, lac pernease and CcdB gyrase. Lac permease is required for entry into E. coli cells of the poison o-Nitrophenyl-β-D-thiogalactoside (“TONPG”). Regulation of the expression of lac permease using gene regulator fusion proteins can, in the presence of TONPG, determine the viability and growth of bacterial cells. See, J. Miller A Short Course in Bacterial Genetics, Cold Spring Harbor Press (I 992). Expression of the CcdB gyrase poison is lethal to E. coli cells, and hence gene regulator fusion protein influence over expression of CcdB can be used for drug resistance positive selections. See Bernard et al., J. Mol. Biol., 226:735-745 (1992).

The use of gene regulator fusion proteins for proactive determination of drug resistant genotypes of chemotherapeutic target proteins in accordance with the methods of the present invention has a number of advantages over existing methods. First, using the method of the present invention, target protein (e.g., protease), variant libraries can be screened for drug resistance after less than sixteen hours of cell growth in contrast to currently used cell culture selection methods which require several months of cell passaging before drug resistant mutants arise. While the RT-ELISA methods for determination of drug resistant genotypes described in PCT International Publication No. WO96/08580 are much quicker than cell culture selection methods, the RT-ELISA methods still require more labor than the gene regulator fusion protein methods according to the present invention.

In addition, the methods of the present invention allow the scientist to use common, readily available bacterial strains and laboratory reagents which are relatively inexpensive. Moreover, very little labor is required to use the bacterial strains and reagents. This is markedly in contrast with the resources required for maintenance of viral infected cell culture over the durations required for effective discovery of drug resistance using the cell culture selection methods. Similarly, the gene regulator fusion protein methods described herein are also less expensive than the RT-ELISA method.

The methods of the present invention are also much safer than the cell culture discovery methods, as the methods of the present invention use bacterial gene regulator fusion proteins and do not require handling of whole, potentially infective, viruses.

Additional benefits of the present invention over the existing methods include, but are not limited to, the following:

(1) Design of positive selections: As described above, the present invention can use gene regulator fusion proteins to control the expression of toxic genes which will allow direct or positive selection of E. coli cells which express drug resistant variants of the target protein.

(2) Uncomplicated interpretation of results: Using gene regulator fusion proteins in accordance with the present invention, the selected, drug resistant target protein variants are easily analyzed by DNA sequencing, and the gene can be transferred to a fresh vector and bacterial cell to insure that the protease mutations of that gene do indeed confer the resistance reported. Variables such as induction of expression of the fusion protein and of reporter protein are completely under the control of the researcher. In contrast, using cell culture selection, variations in highly complex mammalian cultured cells as well as variations in whole virus genomes contribute to the determination as to which cells survive exposure to chemotherapeutic agents.

(3) Overcomes “blind spots” of cell culture selection/discovery:

(a) One potential blind spot of cell culture selection is that some protease mutations may compromise viral viability, since a subset of protease drug resistant mutations are expected to compromise viral viability, e.g. the [R8Q] mutation. See Kaplan et al., Proc. Natl. Acad. Sci., 91:5597-5601 (1994); and Ho et al., J. Virol., 68:2016-2020 (1994). If the viability/infectivity detriment is severe, the virus will be prevented from “taking” to cell culture and will therefore not be discovered. However, these types of mutations can be discovered using the methods of the present invention, and such mutations should not be ignored in light of the extreme heterogeneity of clinical viral populations as viral variations either within the protease gene (e.g., [M461] increases viability in culture of the [R8Q] HIV-PR variant) or at other loci can “compensate” for the detrimental mutation.

(b) Another potential blind spot of cell culture selection relates to double mutations where neither single mutation effects drug susceptibility. Use of very large libraries as in the present invention, however, allows microbial discovery of drug resistance conferred by multiple mutations. Multiple mutations (where each of the changes contributes to phenotype) found in cultured cells are generally the result of gradual sequential accrual. For this reason, multiple mutations where neither single mutation confers phenotype, are more likely to be discovered using the methods according to the present invention.

(c) Cell culture discovery requires prolonged passaging starting with a single HIV variant; different HIV variants, however, show different susceptibilities to several potential inhibitors. See D. Richman, Ann. Rev. Pharmacol. Toxicol., 32: 149-164. (1993); and Sardana et al., Biochemistry, 33: 2004-2010 (1994). Using microbial systems in accordance with the present invention, however, can easily allow substitution of HIV-1, HIV-2 or other HIV protease genes as backbones in which to induce mutations.

### EXAMPLE 1

### HIV-PR genotype and HIV-PR inhibitors influence on color of E. coli colonies using a HIV-R-LacI fusion protein expression plasmid/β-Gal reporter plasmid, two plasmid system

Three E. coli strains, each containing a reporter plasmid for expression of β-galactosidase, and a HIV-PR-LacI expression plasmid for expression of a HIV-PR-LacI fusion protein, were tested using the blue/white color assay of the present invention. These strains were designed to be identical except for mutations within the HIV-PR regions, as set forth below:

| PLASMID | HIV-PR TYPE | FUSION PROTEIN |
|---|---|---|
| pL446.1 | native HIV protease (124) | PR-LacI |
| pL447.5 | drug resistant HIV protease (228) | PR-LacI |
| | (contains [M46I, L63P, V82T, & | |
| | I84V] amino acid substitutions) | |
| pL448.2 | inactive HIV protease (164) | PR-LacI |
| | (contains [D25E] amino acid | |
| | substitution) | |

Plasmid pL446.1 contains the native HIV-PR₁₂₄, plasmid pL447.5 contains drug resistant HIV-PR₂₂₈, and plasmid pL448.2 contains inactive HIV-PR₁₆₄. Plasmid pL446.1 expresses a fusion protein containing HIV-PR and the LacI gene repressor. Expression is mediated by the ARAB promoter/operator and expression is induced by addition of arabinose sugar to the growth medium. The plasmid is derived by subbcloning HIV and LacI gene sequences into the vector pAR3. See Perez-Perez, J. and J. Gutierrez, Gene, 158:141-142 (1995).

The map of fusion protein expression plasmid pL446.1 is as follows:

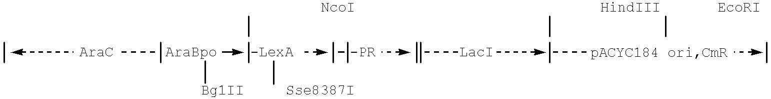

Plasmids pL447.5 and pL448.2 were constructed to be identical to pL446.1 except for a 525 bp DNA segment bordered by the restriction sites BglII and Sse8387I containing the entire HIV-PR gene. The HIV-PR-LacI fusion protein junction is set forth below, and the genotypes of the different HIV-PR variants encoded by these BglII, Sse8387I DNA fragments are demonstrated herein.

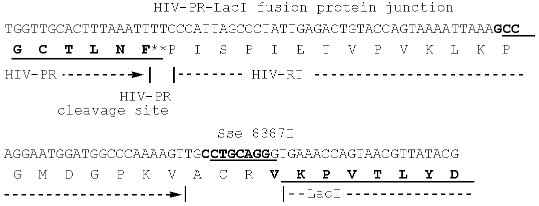

Media plates were prepared by adding to standard Luria Broth Agar, per liter of Luria Broth Agar, 2,000 μl of 100 mg/ml Ampicillin, 800 μl of 34 mg/ml Chloramrphenicol, 1 ml 1M IPTG. Similarly, indicator media was prepared by adding Bluogal (Life Technologies Inc.) to the media, 16.8 ml Bluogal stock (2% in dimethyl formamide) per liter of Luria Broth Agar. In addition, various amounts of 125 mg/ml arabinose and 100 mg/ml indinavir solution in 50% ethanol were added to some media plates as set forth in Table 1. The E. coli strains were then plated onto the indicator media and allowed to grow for about 16 hours. The colors of the resulting colonies on each plate are set forth in Table 1.

TABLE 1
Results of Blue/White Color Assay of E. coli Strains Expressing Native,
Drug Resistant, & Inactive HIV Protease On Indicator Media Containing
Different Levels of Gene Expression Inducer and of Protease Inhibitor

[chemical structure]

W = White;
B = Blue
Arabinose = inducer of expression of the fusion protein
indinavir = CRIXIVAN = MK-639 is the HIV protease inhibitor of Merck & Co.

On indicator media containing the Bluogal (Life Technologies Inc.) calorimetric substrate as well as inducer of expression of the fusion protein, colonies of E. coli containing pL448.2 (inactive HIV-PR) appeared dark blue, indicating a failure to turn off expression of the β-galactosidase gene in the reporter plasmid. In contrast, for E. coli strains containing plasmids pL446.1 and pL447.5, which express native and drug-resistant HIV-PR respectively, the colonies are white, indicating activity of LacI to prevent β-galactosidase expression. When these same three strains are grown on indicator media supplemented with the HIV-PR inhibitor indinavir at various concentrations, pL448.2 (inactive HIV-PR) remains blue but now pL446.1 (native HIV-PR) is also appears dark blue, indicating failure of inhibited HIV-PR to activate LacI. However, the strain containing pL447.5 (drug resistant HIV-PR), remains white, even in the presence of high levels of indinavir, indicating failure of indinavir to inhibit HIV-PR activation of LacI.

This example demonstrates using a high contract blue/white color assay in the methods of the present invention for the identification of E. coli strains which express drug resistant HIV-PR. The basis of the assay is the expression by E. coli of a fusion protein containing HIV-PR and the LacI repressor of gene expression from a first plasmid, the fusion protein expression plasmid. Active LacI repressor turns off expression of another gene in a second plasmid, the reporter plasmid, which encodes β-galactosidase, whose activity is indicated by the processing of a colorless substrate (Bluogal or Xgal) to yield a dark blue precipitable product. Thus, using this system, if the fusion protein expression plasmid contains a drug resistant HIV-PR LacI is activated by the HIV-PR and released from the fusion protein, and the active LacI turns off β-galactosidase gene expression in the reporter plasmid, and hence colonies appear white.

### EXAMPLE 2

### Verifying Authenticity of Method Using PR Variants of Known Genotype

Site directed mutagensis was used to construct HIV-PR gene variants encoding the mutations 1) [D25E] (inactive protease), 2) [M46I+L63P], 3) [M46I+L63P+V82T], and 4) [M46I+L63P+V82T+I84V]. These variant genes were put into fusion protein expression plasmids in the manner set forth in Example 1, and the resulting fusion protein expression plasmids and a β-galactosidase reporter plasmid expression were subcloned into E. coli, which were then plated on indicator media designed to report E. coli having HIV-PR activity as white colonies and E. coli without HIV-PR activity as blue colonies.

Unless otherwise indicated, indicator media used herein was made by adding 2,000 ml 100 mg/ml Ampicillin, 800 ml 34 mg/ml Chloramphenicol, 1 ml 1M IPTG, 5 ml 125 mg/ml arabinose, 5 ml 100 mg/ml indinavir solution in 50% ethanol, and 16.8 ml Bluogal stock (2% in dimethyl formamide) per liter of standard microbiological Luria Broth Agar.

In this example, E. coli cells expressing the HIV-PR-LacI fusion proteins were replica plated onto two media plates, wherein both plates contained indinavir protease inhibitor, and only one plate contained Bluogal (Life Technologies Inc.). In the method of the present invention, β-galactosidase activity is regulated indirectly by activity of the HIV-PR in the fusion protein expression plasmid. All the E. coli cells used were identical and expressed similar HIV-PR-LacI fusion proteins, except that the HIV-PR genotype is different for different colonies.

The plate lacking the color indicator revealed that all the E. coli cells grow similarly on both plates. However, for the plate containing both the protease inhibitor indinavir or MK-639 and the color indicator, only E. coli colonies containing drug resistant HIV-PR variants were white. Through DNA sequencing, it was confirmed that only the white colonies reported on the indicator plate contained HIV-PR variants expressing the resistance-conferring mutations V82T or I84V. Furthermore, the degree of “whiteness” of the colonies containing resistance-conferring mutations is greater for colonies which contained HIV-PR variants expressing the V82T and I84V mutations, than for colonies which contain HIV-PR with the V82T mutation. In fact, the degree of whiteness of colonies correlates well with known Ki values for the resistant genotypes, and accurately ranks, with respect to lowered susceptibility to indinavir, the HIV-PR variants as: [Native], [M46I, L63P], [M46I, L63P, V82T] and [M46I, L63P, V82T, I84V]. The resistance-conferring mutations identified in the white colonies obtained in this example exhibit a strong correlation with mutations known to contribute to clinical resistance to indinavir.

### EXAMPLE 3

### Construction of L434 Library of Randomly Mutagenized HIV-PR Variant Genes

A library of HIV-PR variant genes containing dispersed mutations within the HIV-PR coding region was contstructed. This library, designated L484, contains the “backbone” protease gene polymorphism L63P which is found in a high proportion of clinical samples and in combination with other mutations, is associated with heightened levels of drug resistance. The library variant genes are expressed in E. coli as fussion proteins with a LacI repressor using fusion protein expression plasmids made as set forth in Example 1, and the E. coli also contains a β-galactosidase reporter plasmid such that protease activity is reported on indicator media by colony color.

Several methods are available for introduction of randomly distributed mutations within a defined DNA region. In the present case, a manganese ion induced error prone PCR method was used. See, Cadwell, C. and G. F. Joyce, PCR Meth. and Applications, 2:28-33 (1992). Error prone PCR was used to amply a portion 525 base pair DNA segment containing the entire HIV-PR gene. The restriction sites BglII and Sse8387I were added to the PCR primers to allow replacement of native sequences of pL446.1 (see Example 1 above) with the mutagenized DNA segments.

Approximately 2,000 E. coil colonies containing fusion protein expression plasmid/reporter plasmid vectors were grown on color indicator media containing indinavir. Six white colonies were then selected from the background of 2,000 blue colonies. Four of these were comparable in degree of whiteness to a control colony expressing the highly resistant HIV-PR variant [M46I L63P V82T I84V]. Two others were somewhat bluer (indicating higher protease drug susceptibility). The DNA sequences of the six selections were determined as shown in Table 2.

TABLE 2
Genotypes of HIV-protease variants selected as drug resistant using
the HIV-PR-gene regulator fusion protein reporter selection

| | Library | | genotype (all | |
|---|---|---|---|---|
| | (All variants | | contain the L63P | |
| | contain the | | polymorphism) | |
| | resistance | colony | Bold are | |
| Isolate | enhancing | color | associated with | |
| identi- | polymor- | W = white | clinical | |
| fication | phism L63P) | B = blue | resistance | |
| Control | | W | M46I V82T | clinical |
| resistant | | | I82V | resistance to |
| (pL-447.5) | | | | indinavir and |
| | | | | (all) other |
| | | | | HIV-PR |
| | | | | inhibitors |
| +E,uns WB2a | L484 (L63P) | W/b | M46L T74S | |
| WB4a | L484 (L63P) | W | I3V I84V | I3V is a |
| | | | | naturally |
| | | | | occurring |
| | | | | polymorphism. |
| | | | | I84V is critical |
| | | | | to high level |
| | | | | resistance to |
| | | | | indinavir. |
| WB6a | L484 (L63P) | W | K55I | |
| WB9a | L484 (L63P) | W | E21Q +E,uns M46T | V82 substit- |
| | | | +E,uns V82F | utions are |
| | | | | amoung the most |
| | | | | frequently |
| | | | | found to be |
| | | | | associated with |
| | | | | resistance to |
| | | | | indinavir, |
| | | | | Ritonavir and |
| | | | | other protease |
| | | | | inhibitory |
| | | | | drugs. |
| WB9b | L484 (L63P) | W | K45I +E,uns M46I | The K45I M46I |
| | | | F53I | combination is |
| | | | | found |
| | | | | transiently |
| | | | | in one of |
| | | | | Merck's |
| | | | | patients which |
| | | | | is resistant to |
| | | | | indinavir. (The |
| | | | | virus is thus |
| | | | | potentially |
| | | | | viable). |
| WB10 | L484 (L63P) | W/b | +E,uns M46I | Similar to |
| | | | | WB2a |

Condra et al. (J. Virol., 70:8270-8276) present comprehensive DNA sequence analysis of patients who developed viral resistance to indinavir. All of the 29 resistant viral isolates examined displayed alterations of positions M46 (to I or L) and/or V82 (to A, F or T). In addition, I84V is strongly associated with high levels of indinavir resistance. The results set forth herein clearly demonstrate that the method of the present invention, which uses a highly simplified bacterial expression colony color screen, independently identifies these same resistance conferring mutations as are found in the clinic.

The embodiments of the present invention described above are intended to be merely exemplary and those skilled in the art will recognize, or be able to ascertain using no more than routine experimentation, numerous equivalents to the specific procedures described herein. All such equivalents are considered to be within the scope of the present invention and are covered by the following claims.

The contents of all references described herein are hereby incorporated by reference.

Other embodiments are within the following claims.

What is claimed is:

1. A method for detecting mutations in a target protein that confer resistance to a chemotherapeutic agent or drug directed against the target protein, said method comprising the steps of:
(a) preparing random mutations of the gene for the target protein;
(b) subcloning each of the resulting mutant target protein genes into an expression vector or plasmid to form an extended open reading frame encoding a fusion protein including both the target protein and a regulator protein;
(c) preparing a reporter plasmid containing in proper reading sequence a gene for a reporter protein whose activity is regulated by the regulator protein;
(d) introducing the fusion protein expression plasmid from step (b) and the reporter plasmid from step (c) into bacterial cells by electroporation to form a bacterial expression library;
(e) plating the resulting bacterial expression library onto a suitable indicator media containing an amount of a chemotherapeutic agent against the target protein, and incubating the resulting media plates for a period of time; and
(f) identifying from the resulting colonies those colonies which contain drug resistant target protein based on a reporter mechanism of the reporter protein.

2. A method according to claim 1, wherein the target protein is a protease.

3. A method according to claim 2, wherein the protease is HIV protease.

4. A method according to claim 1, wherein the regulator protein is LacI repressor protein.

5. A method according to claim 3, wherein the regulator protein is LacI repressor protein.

6. A method according to claim 4, wherein the reporter protein is β-galactosidase.

7. A method according to claim 5, wherein the reporter protein is β-galactosidase.

8. A method according to claim 1, wherein the bacterial cells are E. coli cells.

9. A method according to claim 4, wherein the bacterial cells are E. coli cells.

10. A method according to claim 5, wherein the bacterial cells are E. coli cells.

11. A method according to claim 8, wherein the reporter mechanism is a white color.

12. A method according to claim 9, wherein the reporter mechanism is a white color.

13. A method according to claim 10, wherein the reporter mechanism is a white color.
